(19) United States
(12) Patent Application Publication
(10) Pub. No.: US 2013/0280698 A1
(43) Pub. Date: Oct. 24, 2013

(54) RAPID MULTIPLEX LATERAL FLOW ASSAY DEVICE

(71) Applicant: ARIZONA BOARD OF REGENTS, acting for and on behalf of NORTHERN ARIZONA UNIVERSITY, Flagstaff, AZ (US)

(72) Inventors: Catherine R. Propper, Flagstaff, AZ (US); John T. Tester, Flagstaff, AZ (US); Timothy L. Vail, Parks, AZ (US); Niranjan Venkatraman, Flagstaff, AZ (US)

(21) Appl. No.: 13/869,913

(22) Filed: Apr. 24, 2013

Related U.S. Application Data
(60) Provisional application No. 61/637,791, filed on Apr. 24, 2012.

(51) Int. Cl. G01N 33/53

(52) U.S. Cl. CPC G01N 33/5302
USPC 435/5; 422/401; 422/402; 422/403; 422/404; 436/501; 435/287.2; 435/7.1

(57) ABSTRACT

Disclosed herein are devices, systems and methods for simultaneously conducting multiple assays on a liquid sample for the presence of, and/or quantification of, analytes in the liquid sample. The device, which is referred to herein as a multi-assay cartridge or multi-strip assay cartridge (MSC), may be used in a system comprised of the cartridge and a reading device or reader for reading the assays in the cartridge.

### CROSS REFERENCE TO RELATED APPLICATIONS

This application claims priority to U.S. provisional application Ser. No. 61/637,791 filed Apr. 24, 2012, the entire disclosure of which is hereby incorporated by reference.

### FIELD OF THE INVENTION

The invention relates to a method and device and system for analyzing a single sample of an analyte, to detect the presence in the analyte of certain components of interest.

### SUMMARY OF THE INVENTION

Disclosed herein are devices, systems and methods for simultaneously conducting multiple assays on a liquid sample for the presence of, and/or quantification of, analytes in the liquid sample. The device, which is referred to herein as a multi-assay cartridge or multi-strip assay cartridge (MSC), may be used in a system comprised of the cartridge and a reading device or reader for reading the assays in the cartridge.

Nonlimiting examples of samples that may be analyzed using the invention include water, serum, saliva, whole blood, and beverages. Results can be readily incorporated into a database for use in medical or veterinary diagnostics, environmental monitoring, contamination site management decision, remediation technology performance monitoring, and multiple site/multiple remediation technology comparisons.

The MSC device provides a way to simultaneously and automatically identify different assay results from a single sample. It is based on the quantitative analysis of digitized data from rapid immunochromatographic assays. The assay results may be identified via viewing with the human eye, or via an optical image collected from the reader device. The reader device can be any type of automated reader, but is preferably a opto-electronic reader. Still more preferably, the reader device is hand-held and portable.

### BRIEF DESCRIPTION OF THE DRAWINGS

FIG. 1 illustrates a perspective view of the exterior top portion of a multiplex assay cartridge device according to an embodiment of the invention.

FIG. 2 illustrates a perspective view of the exterior bottom portion of a multiplex assay cartridge device according to an embodiment of the invention.

FIGS. 3 and 4 illustrate perspective views of a multiplex cartridge device according to an embodiment of the invention, wherein the cartridge's housing has been disassembled into top and bottom pieces, showing elements in the interior of the bottom piece of the housing.

FIGS. 5 and 6 illustrate perspective views of a multiplex cartridge device according to an embodiment of the invention, wherein the cartridge's housing has been disassembled into top and bottom pieces, showing elements in the interior of the top piece of the housing.

FIG. 7 illustrates a cross-sectional view of the embodiment of the cartridge device shown in FIGS. 1-6.

FIG. 8 illustrates a schematic illustration of a portion of a multiplex assay cartridge device according to an embodiment of the invention, showing a part of the device as transparent so as to show some of the underlying elements in the device.

FIG. 9 illustrates a perspective view of a single-assay cartridge device according to an embodiment of the invention.

FIG. 10 illustrates a perspective view of a single-assay cartridge device according to an embodiment of the invention, wherein the cartridge's housing has been disassembled into top and bottom pieces, showing elements in the interior of the bottom piece of the housing.

FIGS. 11 and 12 illustrate a perspective view of several single-assay cartridge devices joined together according to an embodiment of the invention.

FIG. 13 is a front view, and FIGS. 14 and 15 are side views of a single-assay cartridge device according to an embodiment of the invention.

FIG. 16 is an illustration of a perspective view of a reader according to an embodiment of the invention.

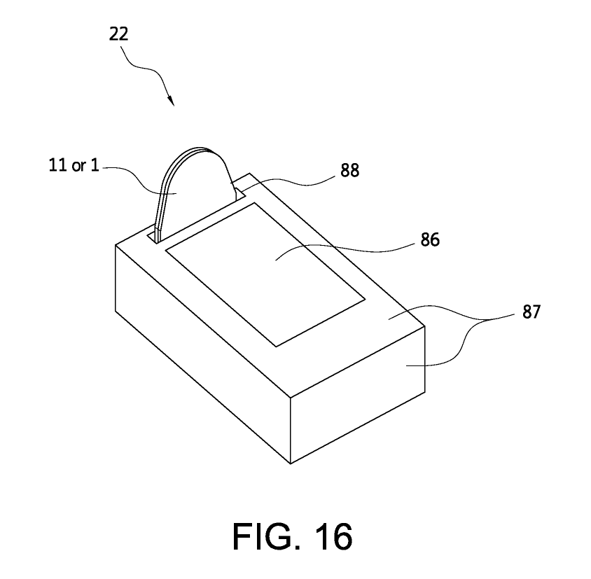

### DETAILED DESCRIPTION OF THE INVENTION

The cartridge device, system and method of the invention may be used in a variety of situations wherein a rapid diagnostic panel of tests that relies on testing liquids is desirable. The invention provides a way for the user to simultaneously test a single sample of whatever fluid is to be analyzed, via multiple assays. The assays used will depend upon the particular chemicals, biological components (e.g., antibodies), etc. that the user is seeking to determine whether the sample contains. A single MSC can contain multiple assays that are identical (to confirm accuracy of the test results), or each assay can be a different one.

The invention provides the ability to gain near real-time results from multiple tests conducted simultaneously, wherein the tests are as sensitive or nearly as sensitive as expensive laboratory systems such as gas or liquid chromatography. The invention has the further advantage that it eliminates the necessity to transport the samples to be tested to a laboratory. In addition, the cartridge devices described herein are easy to handle and use.

The invention is preferably used with lateral flow assays, which are known to those of ordinary skill in the art. Lateral flow assays may indicate the detection of a component of an analyte via a change in signal intensity, or other means, for example, via color change in the visible light spectrum. Lateral flow assays typically employ an assay strip.

Non-limiting examples of situations or environments wherein such rapid diagnostics are desirable include water testing in the field in both emergency (such as post-natural disaster or other disasters) and non-emergency situations; biological sample testing in emergency medicine, triage or disaster medicine, and in non-emergency settings; veterinary medical testing; and in factories and food processing plants such as to monitor industrial processes,

Non-limiting examples of analytes which may be tested using devices, systems and methods of the invention include water, and biological fluids (e.g., sputum, mucous, urine, saliva, blood, semen), chemicals, food beverages, and other liquids. For example, an environmental water sample obtained from a natural body of water, tap water, a water treatment facility, etc. may be tested for various components and/or conditions, such as pH, chemicals, microbes (e.g., algae, bacteria, fungi, viruses), hormones, or a person's or animal's urine sample could be tested for the presence of various components and/or conditions, such as pH, hormones, glucose, and microbes (e.g., bacteria, fungi, viruses). The invention could also be used for analyzing other types of liquids, such as beverages, foods and medicines. For example, milk, fruit juices, sodas, medicines, etc. could be analyzed using the device of the invention.

In a specific embodiment of the invention, the device is used to rapidly analyze water samples to detect the presence of contaminants known as endocrine-disrupting compounds, that impact reproduction and development of living organisms. Water resources throughout the world are becoming contaminated with these and other human-produced compounds that mimic the actions of naturally occurring hormones.

The liquid sample must be of relatively low viscosity in order to be analyzed in the device of the invention. Therefore, it may be advantageous to lower the viscosity of certain liquids to be analyzed, by diluting the liquid (such as by combining with a buffer solution). For example, a biological sample such as sputum may, prior to analysis with this device, be diluted in a buffer solution.

Optionally, the system disclosed herein is also provided with means to quantify the component(s) being detected. In such an embodiment, the system will be provided with a calibration/reference component.

A first embodiment of the device is a disposable cartridge comprising two or more different assays. In a preferred embodiment, the cartridge comprises five different assays. In still yet another preferred embodiment, the cartridge comprises seven different assays. The device is used by introducing a liquid sample to the cartridge, wherein the sample is subjected to analysis. Preferably, the cartridge will contain lateral flow assays. Even more preferably, the cartridge will contain lateral flow assay strips.

The first embodiment of the device or cartridge for simultaneously conducting multiple assays on a liquid sample is comprised of the following components:

(a) a housing;

(b) an inlet in the housing for introducing the liquid sample into the housing;

(c) a diversion dam within the housing and in communication with the inlet;

(d) a diversion dam channel within the housing and in communication with the diversion dam;

(e) a first assay chamber and a second assay chamber within the housing, wherein the first assay chamber comprises a first assay for detecting the presence of an analyte in the liquid sample, and the second assay chamber comprises a second assay for detecting the presence of an analyte in the liquid sample; and

(f) a first flow channel and a second flow channel, wherein the first flow channel is in communication with the diversion dam channel and the first assay chamber, and the second flow channel is in communication with the diversion dam channel and the second assay chamber.

The housing is typically comprised of a hard plastic material, but other materials are possible.

The device is used by introducing into the inlet a liquid sample to be tested. In a preferred embodiment, the liquid sample is about 1 ml in volume, although smaller and larger volumes may be used.

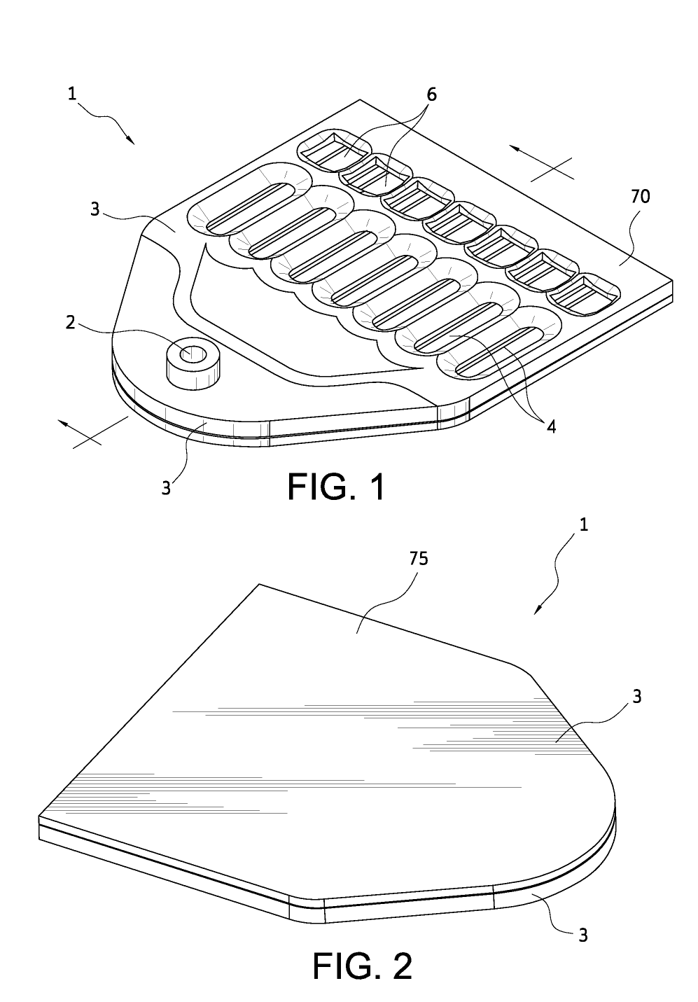

FIGS. 1 through 6 illustrate an embodiment of the invention described above comprising seven assays. In this embodiment, the cartridge device is comprised of the following components:

(a) a housing 3;

(b) an inlet 2 in the housing for introducing the liquid sample into the housing;

(c) a diversion dam 10 within the housing and in communication with the inlet;

(d) a diversion dam channel 12 within the housing and in communication with the diversion dam;

(e) seven separate assay chambers 30 within the housing, wherein each assay chamber comprises an assay for detecting the presence of an analyte in the liquid sample; and

(f) seven flow channels 20, each in communication with the diversion dam channel and a different assay chamber.

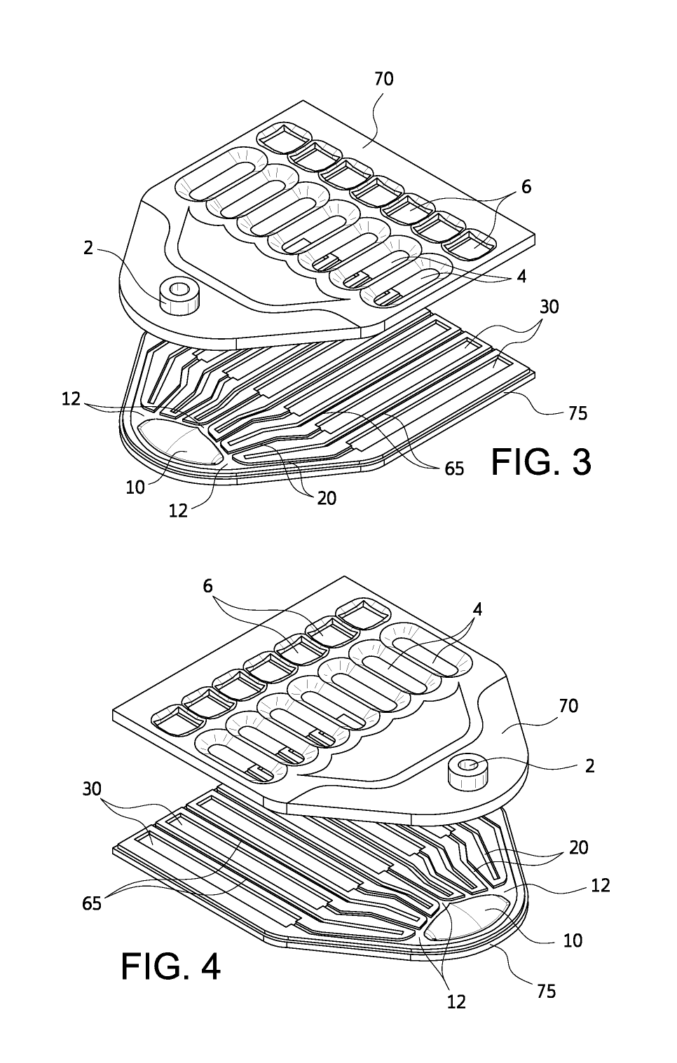

FIG. 1 illustrates a perspective view of the exterior top portion, and FIG. 2 illustrates a perspective view of the bottom, of the cartridge device 1 according to an embodiment of the invention. The term “bottom” refers to the side of the cartridge that in use is placed on a table or other flat surface, so that the “top” faces the user. The housing 3 is comprised of a top piece 70 and bottom piece 75.

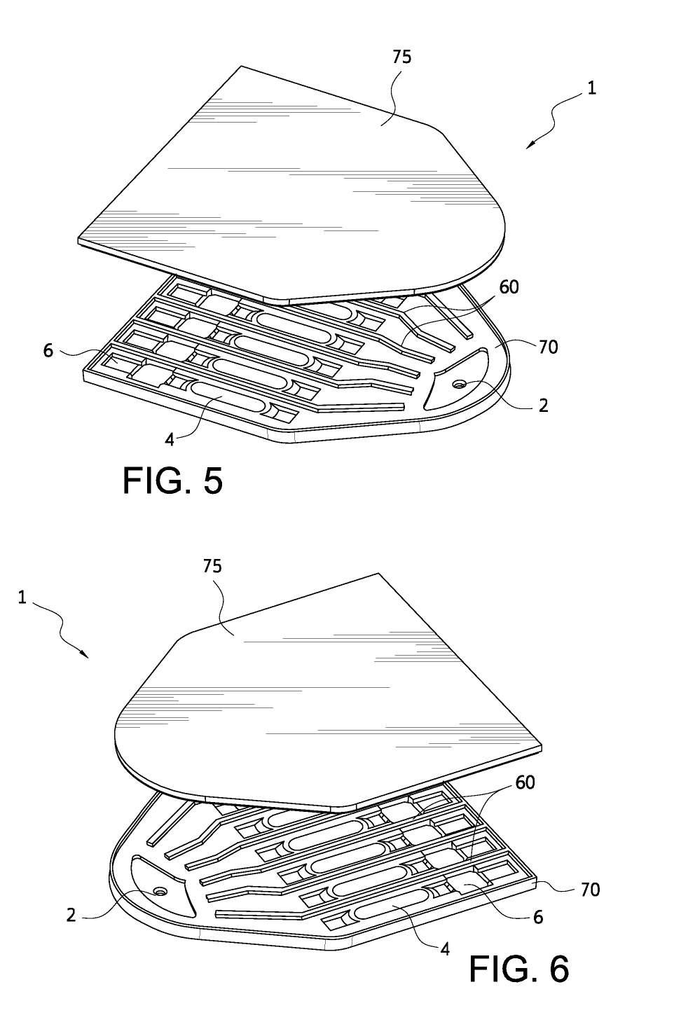

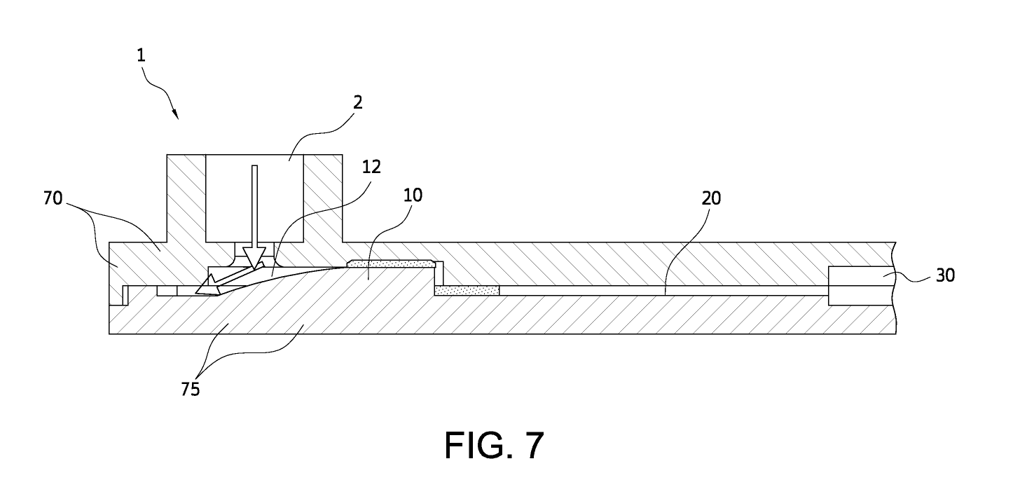

FIGS. 3 and 4 are additional perspective views of the cartridge, with the top piece 70 and the bottom piece 75 disassembled from one another, illustrating the interior components of the cartridge device 1 according to the embodiment of the invention shown in FIGS. 1 and 2. FIGS. 3 and 4 show the components that would be visible if the housing 3 was disassembled and the top piece 70 and bottom piece 75 were maintained in the same orientation in which the device 1 is normally used. In contrast, FIGS. 5 and 6 show the components that would be visible if the device 1 was turned 180 degrees (i.e., turning the device upside down) and the housing 3 was disassembled.

Although indicium viewing windows 6 are shown in FIGS. 1 through 6, these windows 6 are actually optional for this embodiment.

The exterior shape of the embodiment of the cartridge shown in FIGS. 1 through 6 and other figures are merely examples of the possible exterior shapes of the cartridge. For example, the cartridge could have a rectangular overall shape.

In use, the liquid sample to be tested is introduced into inlet 2 in the housing 20. The user can view progress and results of the assay through the seven assay view windows 4. Seven optional indicium viewing windows 6 are illustrated in FIGS. 1, 3 and 4.

Each assay chamber 30 may further comprise an assay viewing window 4 in the housing for reading the progress or results of the assay. For example, the results of the test may be a color change, appearance of lines or other indicia, etc. The assay viewing window 4 is typically simply a hole in the housing 3, so that the assay is exposed to the air. However, in certain applications, such as for testing liquids that may contain, or themselves be, hazardous materials, the window may have a transparent shield comprised of hard plastic, a film, glass or other material to permit the user to view the progress and results of the assay, while preventing the contents of the sample within the assay chamber, and the assay materials themselves, from moving out of the assay chamber to expose the user and/or being touched by the user.

Optionally, in this embodiment, corresponding to each assay chamber 30 there may be an indicium viewing window 6 in the housing 3 for viewing a machine-readable indicium associated with the assay. The indicium may be located on the assay itself, or may be located on the exterior of the housing. For example, for a lateral flow assay, the indicium may be printed directly on the assay strip, or a printed label may be placed on the assay strip. Cartridges that are obtained by the user pre-loaded with specific assays may have the indicium printed onto the housing, rather than directly on the assay strip or on a label.

The indicium is preferably bar code, such as a QR code or a UPC code. Most preferably, the indicium is readable by a machine. Non-limiting examples of machines include optical readers. Specific non-limiting examples of readers are CCD cameras and smart phones. It is contemplated that other technologies currently known or to be developed in the future may be used to read the indicium.

The reader will preferably have the capability of simultaneously capturing an image of the assay results shown in the assay view window 4, and the indicium associated with the assay.

In the aforementioned embodiment, the device 1 is a cartridge that is pre-loaded with multiple assays. In this embodiment, the cartridge is equipped with a receptacle for receiving the analyte sample, and channels or tubes though which the analyte may travel from the receptacle to the assays.

Referring to FIGS. 1 through 6, in this embodiment the cartridge device 1 is constructed of two main parts, a top piece 70 and a bottom piece 75, which are fastened together. Top piece 70 and bottom piece 75 are preferably provided with mating parts on at least their respective outer edges, said mating parts rendering the top piece 70 and bottom piece 75 capable of locking together without adhesives or other means. In yet another embodiment, the MSC device 1 is comprised of top piece 70 and bottom piece 75 which are fastened together with the assistance of tape, glue, other adhesives or other means.

In the embodiment illustrated in FIGS. 1 through 6, the cartridge device 1 is equipped with a single sample inlet 2 that is in communication with a diversion dam 10, which in turn communicates, via diversions channel 12, with multiple flow channels 20. The flow channels 20 are in turn each in communication with a different assay chambers.

During use, the MSC device 1 is preferably, but not necessarily, placed on and maintained with its bottom piece 70 in contact with a substantially horizontal surface, with the sample inlet 2 facing upward, and each flow channel 20 substantially horizontal to the surface on which the device is placed. Alternately, the MSC device 1 can be maintained in a substantially horizontal position by the user or another mechanism, without it being placed on or remaining on a horizontal surface or substrate.

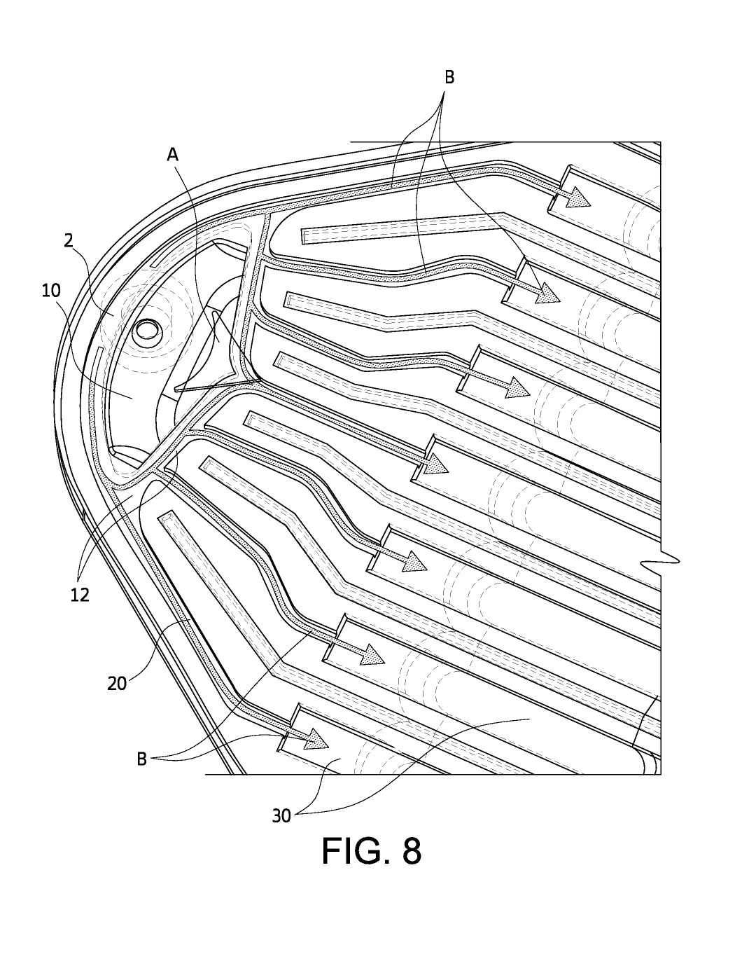

FIG. 7 illustrates a cross-sectional side view of a preferred embodiment of a cartridge device as shown in FIGS. 1 through 6. FIG. 7 shows a top-to-bottom “cut” through the sample inlet 2 and through the diversion dam 10, said cut made through the top piece 70 down through the bottom piece 75, and running from the sample inlet end 4 of the device 1 to the opposite end 5 of the device 1. Also shown in this figure is the diversion dam channel 12, flow channel 20 and assay chamber 30.

The cartridge device 1 has multiple assay chambers 30, and within each chamber 30 is a particular assay strip (not shown). Each assay chamber 30 opens to at least one flow channel 20. Typically, each assay is a lateral flow assay.

The diversion dam 10 forces the analyte sample to divide into substantially equal portions and to travel through the flow channels 20 towards the assay chambers 30. In FIG. 7, the two arrows at the left side of the drawing represent the direction of flow of the liquid sample being analyzed. The larger arrow at the top of the drawing represents the introduction of the liquid sample into the device 1 via inlet 2 onto the diversion dam 10. The smaller arrow on the left side represents the direction of flow of the sample from the diversion dam 10 into the diversion dam channel 12. As shown in FIG. 1, the diversion dam channels are open to and communicate with the flow channels 20.

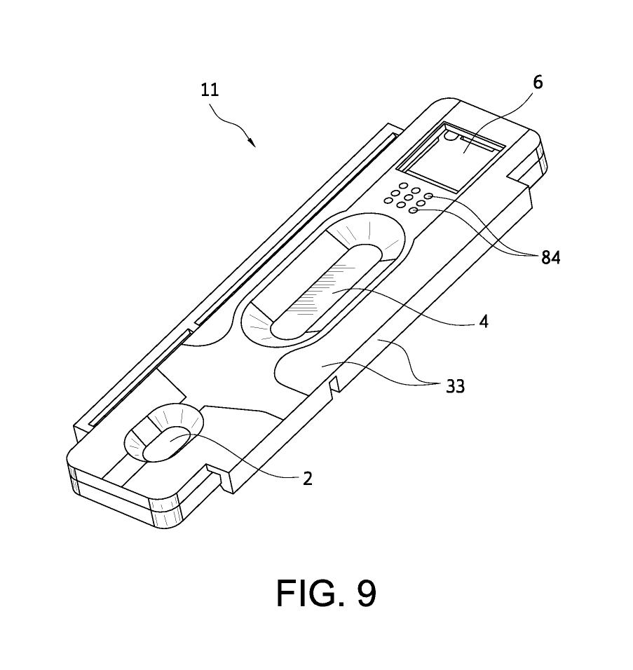

FIG. 8 is a schematic “X-ray” illustration of the embodiment of the cartridge device 1 that is shown in FIGS. 1 through 6. In FIG. 8, the top piece 70 is at least partially transparent, rendering some of the internal components of the device visible. FIG. 8 shows a close up of the portion of the device 1 closest to the sample inlet 2, and illustrates via arrows A and B the flow of the liquid sample in the device, after the sample is introduced via the inlet 2. The liquid sample flows, as illustrated by arrow “A”, from inlet 2, across/over the diversion dam 10 and into the diversion dam channel 12, and then, as illustrated by the multiple arrows “B”, through the flow channels 20 into the assay chambers 30. Illustrated in FIG. 8 is sample inlet 2, diversion dam 10, diversion dam channel 12, flow channels 20, assay chambers 30 (partially shown) and assay view windows 4.

The MSC cartridge device 1 is operated as follows: it is placed in a stationary position, preferably in a horizontal position so that the flow channels and assay chambers are substantially or completely parallel to the horizon. Then, the liquid to be analyzed (e.g., water, blood, urine, etc.) is introduced into the device 1 via the sample inlet 2. The liquid sample may be introduced using a pipette, dropper, syringe, or other suitable means. The liquid sample then travels to the diversion dam 10. The diversion dam 10 is sloped; as the liquid sample makes contact with the diversion dam, the sample flows down the sloped portion of the diversion dam, causing the liquid sample to be substantially evenly distributed to the diversion dam channel 12 and then to the flow channels 20 via gravitational flow. Thus, the diversion dam 10 essentially separates the liquid sample into multiple paths, forcing the liquid into separate flow channels 20. The flow channels 20 are of substantially equal volume, such that the sample is substantially simultaneously split so that it flows through each channel toward each assay chamber 30, into contact with the assay in that chamber 30. The liquid sample flows from the sample inlet 2 through the flow channels 20, to the assay chambers 30 by the force created by expelling the sample into the inlet 2 via pipette, syringe, etc., and/or via capillary flow.

The dimensions (e.g., size, shape, proportions) of the diversion dam channel 12 and the flow channels 20 may vary for different embodiments of the device 1, depending upon various factors, including but not limited to the material that the device is made of (due to different materials having differing coefficients of friction). In other words, the cross-sectional area of the channels in a particular device is preferably designed so that the flow of sample liquid is proportionally sent to each channel 20 from the sample inlet 2.

It is important that each assay chamber 30 is isolated from one another, so that reagents from one assay do not spill over, absorb or otherwise contaminate another assay. FIGS. 5 and 6 illustrate locking ribs 60 on the inside of top piece 70. FIGS. 3 and 4 illustrate locking slots 65 on the inside of bottom piece 75 which mate or engage with locking ribs 60 when the top piece 70 and bottom piece 75 of the housing 3 are secured together. Thus, preferably, when the MSC cartridge device 1 is assembled and the locking ribs 60 and locking slots 65 are engaged or mated, they provide a barrier to prevent the liquid sample and any reagents in the assay from moving from one assay chamber 30 to another.

The above-described embodiment is typically a single-use disposable cartridge, which comes with a pre-determined number and kind of assays.

In yet another embodiment of the invention, the housing 3 of the cartridge 1 may be opened to remove the existing (and perhaps used) assay strips, and new (unused) assay strips inserted in place thereof. The housing 3 is then closed, and the cartridge 1 may be re-used to test another sample. In this re-usable embodiment, after the pre-existing (and perhaps used) assay strips are removed, it is advantageous to clean the interior of the housing 3 to remove all remnants of the previous sample tested as well as the assay materials. With this embodiment, the user can customize the particular assays used, by choosing from a catalog of numerous different assays, each assay which is designed to detect the presence and/or quantity of a specific analyte, and inserting the assay of interest into the cartridge assay holder and/or into the assay device.

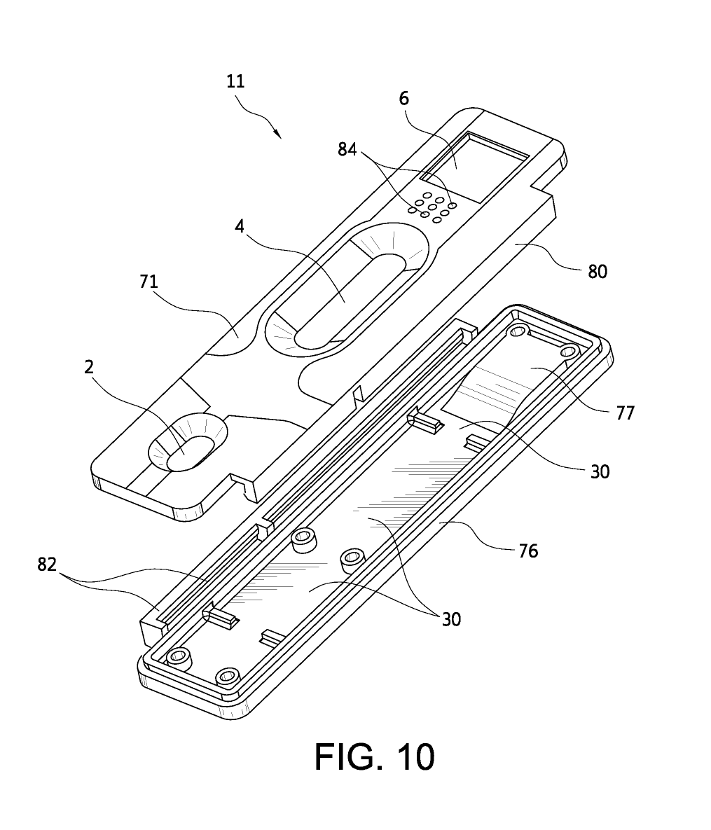

In still yet another embodiment, as illustrated in FIGS. 9 through 15, each cartridge device 11 is comprised of only one assay, and multiple cartridges 11 may be joined together (either by the user or may be pre-joined together) to essentially create a custom analysis tool comprising multiple different types of assays to test for the presence of a variety of different analytes. In other words, each cartridge would have its own housing 33 contain an assay for a particular analyte, and two or more cartridges of the desired assays could be joined together to form a user-defined suite of tests.

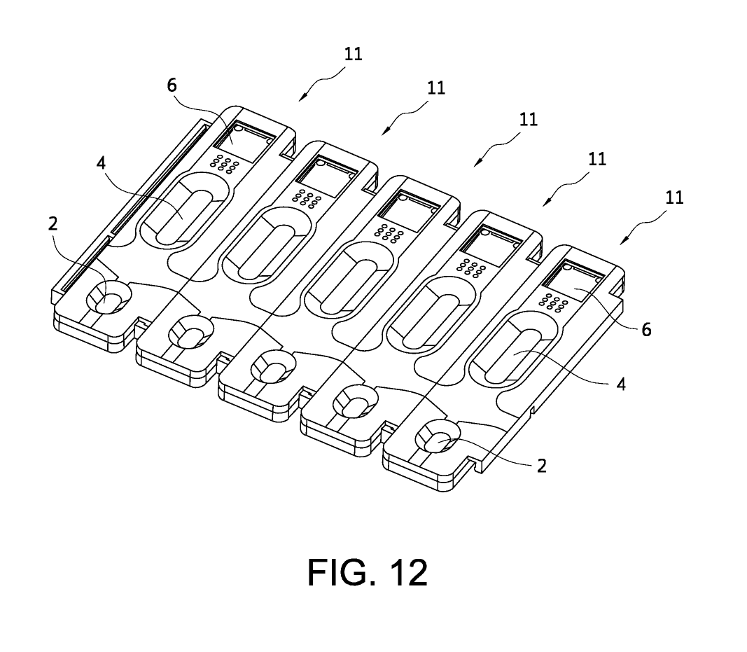

FIGS. 9, 10, 13, 14 and 15 each illustrate one single-assay cartridge device 11. FIGS. 11 and 12 illustrate how multiple single-assay cartridge devices 11 can be joined together to form what is essentially a unitary custom analysis tool.

The means to join the device (the cartridge) to a second device (another cartridge) can be any mechanism that permits the housings of two cartridges to be joined together. For example, the single-assay cartridges have the ability to snap and/or slide securely together. In a preferred embodiment, the cartridge housings 33 are secured to one another by providing the housings with grooves and/or slots, and corresponding projections. The projections mate or otherwise engage with the grooves or slots, in order to attach the cartridges relatively securely to one another.

Preferably, the cartridges are secured to one another in a side by side fashion, in a single row. This permits the user to view the assay view window 4 on each cartridge, and so that a machine reader can capture both the results in the assay view window 4 and the indicia (identifying information) in each indicia viewing window 6

In a variation of this embodiment, not shown in the drawings, a holder is provided, into which single-assay cartridges 11 may be inserted. Preferably, the cartridges are inserted in a side-by-side manner, so that the user may view the assay view window on each cartridge, and so that a machine reader can capture both the results in the assay view window 4 and the indicia (identifying information) in each indicia viewing window 6. The single-assay cartridges may be inserted into the holder by the user, or may be pre-inserted by the manufacturer or supplier of a device according to this embodiment.

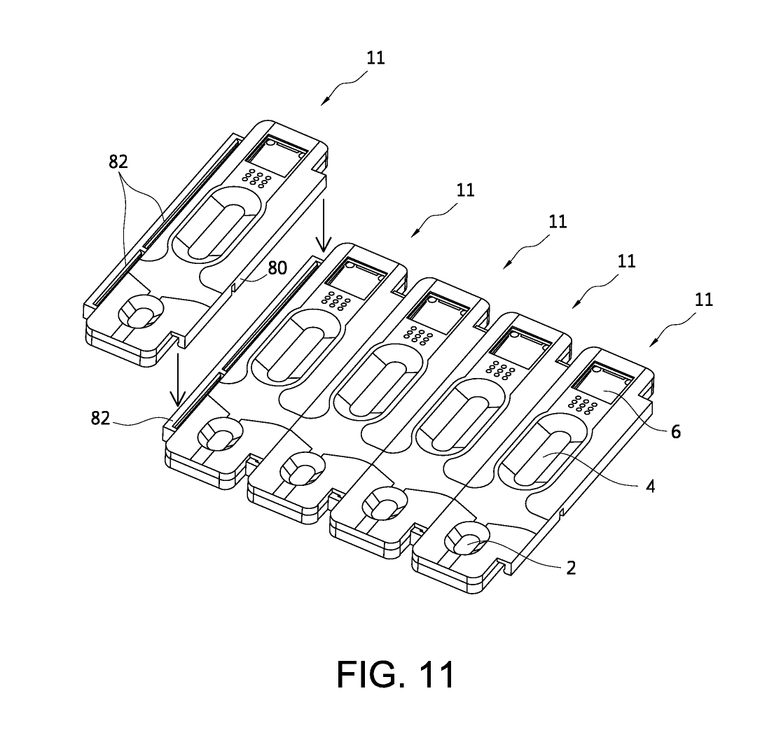

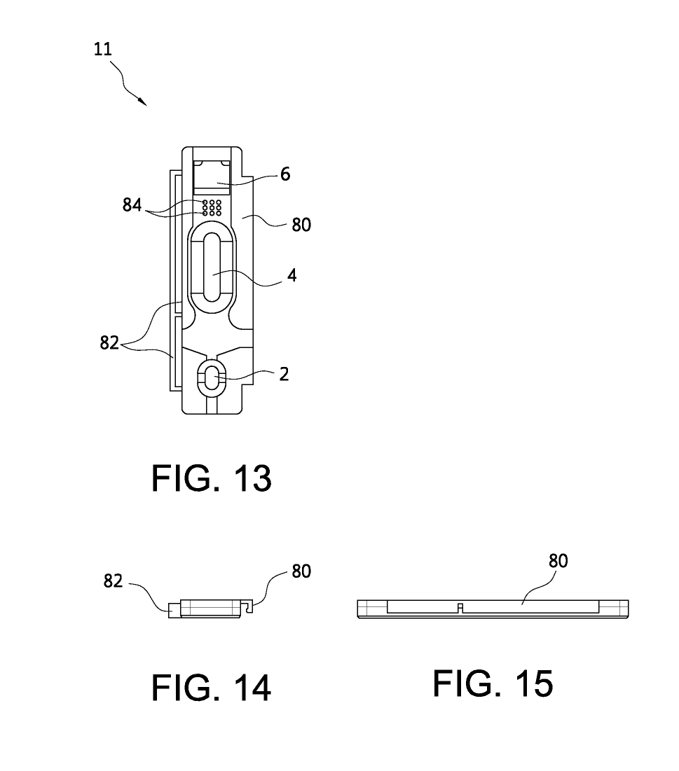

One example of a joining mechanism includes housings having male and female interlocking projections that snap and/or slide into one another. Thus, in an exemplary embodiment, the means to join the single-assay device 11 to a second single-assay device 11 comprises projections on two opposite and substantially parallel sides of the housing 33, said projections capable of interlocking the housing with housing on a second device. As shown in FIG. 10, bottom piece 76 has a female slot 82 on one of its sides, and top piece 71 has a corresponding male projection 80 on is corresponding side. The projection 80 on one cartridge may be inserted into and snapped onto a second cartridge's slot 82, as illustrated in FIG. 11. FIGS. 13 through 15 further illustrate slot 82 and male projection 80.

Shown in FIGS. 9, 10, 11, 12 and 13 are optional air holes 84 in the top piece 71, to permit the release of air from the assay chamber as the liquid sample wicks its way into the assay strip.

The devices according to this single-assay embodiment would comprise

(a) a housing 33;

(b) an inlet 2 in the housing for introducing the liquid sample into the housing;

(c) an assay chamber 30 within the housing comprising an assay for detecting the presence of an analyte in the liquid sample; and

(d) means to join the device to a second device.

In addition, the assay chamber 30 may further comprise an assay viewing window 4 in the housing 33 for reading the progress or results of the assay. Preferably, each assay chamber 30 further comprises an indicium viewing window 6 in the housing 33 for viewing a machine-readable indicium on the assay. The indicium identifies the nature of the assay, i.e., the substance or compound that is being tested for. As with the other embodiment, the indicium may be a bar code.

In the embodiment shown in FIGS. 9 through 15, and as illustrated in FIG. 10, there is at the end of cartridge 11 (the end that is distal the inlet 2), a raised area 77 in the interior of bottom piece 76, which functions to hold in place within the assembled cartridge 11 the assay strip (not shown) that is placed within the bottom piece 76. Alternatively, instead of raised area 77, the top piece 71 could have a raised area in its interior that projects down against the assay strip, to secure the strip in place against the interior of bottom piece 76. In yet another alternative embodiment, indicium viewing window 6 could be structured in a similar manner to inlet 2 and assay viewing window 4, so as to have its interior perimeter extending into the interior of the cartridge 11. In this alternative embodiment, the perimeter of the window 6 that extends into the interior of the cartridge would apply pressure to the assay strip, to maintain the assay strip in place within the cartridge.

The assay results are in some instances able to be ascertained by the naked eye, similar to the popular home pregnancy tests or other presence/absence tests which use visual indicators that are apparent to the human eye without use of microscopy or other means. As shown in the drawings, the cartridge devices of the invention may be provided with assay view windows 50 so that the user can read visually-indicated test results.

In other instances, the assay results are not visible to the naked eye, and therefore must be “read” using a reader machine, such as one that picks up fluorescent signals, infrared signals, etc. In such case, the cartridge containing more than one assay strip, or the group of cartridges secured together, or the group of cartridges placed together in a master holding device) is preferably read substantially simultaneously or consecutively by a reader device.

In preferred embodiments of the cartridges of the invention, each assay is associated with a machine-readable code, more specifically an optically-readable code, to identify the assay. For example, a bar code may be physically located on the assay strip or on the cartridge itself. However, preferably the optically-readable code is placed directly on the assay strip, either by printing onto the strip or by printing the code onto a label on the strip. If an optically-readable code is used, the cartridge 1 or cartridge 11 is provided with indicium viewing windows 6, as shown in FIGS. 1 through 6 and 9 through 13.

In a preferred embodiment of the invention, the reader comprises a CCD camera with a very short focal length that is capable of taking a field of view photograph of the cartridges according to the invention. In a specific example of this preferred embodiment, the CCD camera's focal length is about 1 inch, and the field of view is 6 inches×6 inches. The multi-assay cartridge according to the invention (or multiple single-assay cartridges according to the invention) is inserted under the CCD camera, which takes a high resolution color image of the assay strips and stores the image in a storage device, such as in a flash drive. The data in the storage device is then accessed by the DSP processor. An image processing algorithm programmed into the DSP Processor (such as Texas Instruments TMS 320), which analyzes the strips for the control lines and intensity of the data test lines. The algorithm also identifies the location of the strips in the image of the cartridge. The measurements to be performed for each of the strips are: (a) distance between the control line and the test line, and (b) the intensity of the test line in a numerical ratio to the control line. Alternatively, after the sample is introduced to a cartridge device according to the invention, and the assay reaction occurs, the cartridge can be sent to a facility or location having a machine to read the results.

Alternatively, in a preferred embodiment, the user of the device utilizes a hand-held reader, such as a smart phone or other electronic handheld device, to “read” and/or interpret the results of the assay. This is particularly useful when conducting environmental studies out in the field, in locations remote from analytical laboratories. The hand-held reader may read and analyze the test results, or alternatively may only read the test results and transmit data regarding it to another device (such as wirelessly transmit it to a computer located across the globe) which analyzes the data and transmits the results of the analysis to the hand-held reader for the user to view.

A hand held reader is also preferably used in situations where the results of one or more of the assays are transitory (i.e., the results are only accurate if read within a certain time after the sample analyte is analyzed). For example, water quality tests in the field, and medical diagnostic tests in locations that are geographically remote from where the sample is collected, and/or in triage situations where a particular patient's biological fluids are tested and the results can be immediately (such as from the patient's bedside) uploaded for receipt, storage and/or further analysis at a remote location.

The hand-held reader may be a smart phone, which has been supplied with software application that collects/captured the images produced by each assay. The images so captured may then be transmitted via the smart phone by various methods, such as by direct wireless communication, by email, by texting (SMS or MMS).

In yet another embodiment, the images of the assay may be stored in the user's smart phone or similar device for permanent archival reference, and/or downloaded from the phone to a computer and/or other storage device.

In still yet another embodiment, the smart phone or similar handheld device contains software which interprets the results and notifies the user of the results.

In still yet another embodiment, the invention further comprises a stand for holding the user's smart phone and the cartridge device. The stand will hold the cartridge device, as well as the smart phone. The smart phone will be oriented so that its camera faces the cartridge device and is positioned at the appropriate position to capture images of the assay results.

The following is a description of an exemplary embodiment of a reader for use with the devices (cartridges) of the invention. The reader is a portable electronic assay reader data logger, and interpretation interface. The technology platform is designed to provide the user with a near real-time, cost-effective method for water sample analysis for multiple analytes, database building, and contaminant areal mapping.

The reader is designed to accept cartridges according to the invention. An exemplary embodiment of the reader 22 is illustrated in FIG. 15. Shown in FIG. 15 is a touch screen 86 for the user to interface with the reader, a receptacle 88 in the housing 87 for receiving a cartridge device (1 or 11) according to the invention. However, the exterior appearance of the reader (other than its general size) is not particularly important, so long as its internal components function essentially as described herein. As such, the invention includes readers that are not identical or similar in outward appearance to the reader shown in FIG. 15.

In a preferred embodiment, the reader will accept a unit comprising up to seven individual (single-assay) lateral flow assays, and it quantifies each assay, displays the results of each assay, and archives/exports the data to a database server. The assay cartridge with injection port, inserted into the reader, can be seen in FIG. 15 at the top left of the device. The single-use assay cartridge holds up to seven unique lateral flow assays, each specific for a particular analyte of interest. These assays are technologically similar to home pregnancy or blood glucose tests, where a small sample volume (approximately 1 milliliter) is injected into the micro-flow cartridge, and assays are allowed to develop for approximately eight minutes. All reagents are entrained in the assay, so no “hands-on” chemistry is required from the user other than adding a water sample to the cassette.

Physical Specifications: The reader measures approximately 10″×5″×3.25″ (25.4 cm×12.7 cm×8.25 cm) and weighs less than two pounds (900 g). The housing is ABS plastic, is water resistant, and is designed for field use.

Computer: The reader utilizes a BeagleBoard™, which is a 2 watt, 600 MHz, open source software single board computer, running a Linux™ operating system. This hardware and software provides all of the functionality of a standard laptop PC, but is packaged on a compact 3.25-inch board. This compact design is readily interfaced with a wide range of other relevant sensors or peripheral devices (e.g., temperature, pH, or dissolved oxygen probes) through a USB port (not shown).

Application Software: The reader's application software has been developed in Python™ programming language. Python™ was chosen for its relative simplicity, its dynamic programming style, and its pervasive use across a broad array of application domains.

Data Collection: The reader uses a high resolution digital camera to capture assay results (see below). Numerical information is extracted from the images of the individual test strips for analysis. An average color line value is calculated by integration of the entire line (integrated density value; IDV). This provides a numerical basis of comparison between test and control lines on individual assays, regardless of the nature of the analyte being tested. The test and control lines therefore provide a numerical ratio and within-test normalization. This ratio is then mapped through a non-linear fitting function to a concentration value that has been derived from a series of library assays of known concentration for the individual analyte. The fitting function is a non-linear least squares minimization technique.

User Interface: The primary user interface for the reader is a ULDC7, a 7-inch resistive touchscreen. With full Linux™ support, the reader is capable of using a full desktop PC environment graphical user interface, which provides the interface familiarity of a standard laptop PC or tablet. In a preferred alternative embodiment, the main reader application software can be used as a minimal user interface without a desktop environment for data collection. This results in a significant increase in computational performance. The reader application software is highly configurable for a broad range of possible use scenarios. The application can perform data collection and interpretation on a virtually unlimited inventory of immunochromatographic or colorimetric assays, provided that they can be adapted to use in the assay cartridge. We have demonstrated the ability to read commercially available assays for the commonly used broad herbicides atrazine and simazine, at or below the EPA maximum Contaminant Level (3 ppb for atrazine and 4 ppb for simazine), and have created assays for estradiol and ethynyl estradiol.

After assays using a cartridge according to the invention are performed, the cartridge is inserted into the reader. After the appropriate screen is accessed, the reader displays images of individual assays and quantitative information (analyte concentration expressed as parts per 10× or mass of analyte per standard volume, such as μg/ml, as appropriate). Alternatively, the data can be viewed as a histogram of levels of analyte for each analyzed test strip as a graphical comparison. Results of individual analyses can then be saved and exported to a USB drive (attached through the USB port on the reader, which is not shown in FIG. 15), e-mailed, or transferred via wi-fl or Bluetooth to an Internet-linked or Bluetooth-capable computer or server. We anticipate the deployment of multiple readers within and among contamination sites for the development of a large spatial and temporal data set for GIS-based mapping and analysis.

Power: The reader's hardware requires 2 watts of power, and runs off internal rechargeable batteries or 110V AC current. Current battery life before recharging is estimated at 2.5 hours of continuous use.

Typical Use: The user is required to place a cartridge horizontally on a flat surface, add a 1-milliliter water sample to the cartridge with a single-use syringe, allow the assays to run for approximately eight minutes, follow the on-screen instructions if needed, and read/store the results. Once the data have been stored, the user can recall the data or begin a new test with a new cartridge.

The user interface is designed to allow the operator to move throughout the menu space simply, using a series of on-screen prompts. This interface may be readily modified or customized for specific applications.

The invention also relates to a system for simultaneously conducting multiple assays on a liquid sample, comprising the cartridge device and reader described above. Thus described is a system wherein the reader is a hand-held unit comprising a digital camera and processing hardware and software for interpreting and displaying results of the assays. Further, each assay has an associated indicium that identifies the assay.

The invention also relates to a method for utilizing the cartridge devices and the systems described herein. The method is for simultaneously conducting multiple assays on a liquid sample, comprising: (i)introducing a liquid sample into one of the cartridges described herein, and then reading results of each assay. In a preferred method, the step of reading results is performed by a CCD camera or smart phone.

The method may involve testing of a liquid sample selected from the group consisting of a biological sample obtained from an animal, a biological sample from a plant, or an environmental water sample.

Each assay used in any of the aforementioned embodiments may optionally be equipped with a barcode or other optically coded image, so that if an optical imaging reader is used to collect the assay results, the reader will collect the assay result along with the associated coded image. For example, an assay for the hormone estradiol could be “marked” with an optical code, such as a bar code, which the optical reading machine would read and readily identify that the assay results are for estradiol.

Example of a device according to the invention, comprising an ultra-sensitive rapid assay for the substantially simultaneous detection of estradiol (E2) and ethynyl estradiol (EE2). The assay strips can be chosen from those commercially available, or can be produced as follows. A reagent combination is added to an absorbent strip (such as fleece) and is laminated to the test strip. Addition of the analyte/sample (e.g., water to be tested) re-solubilizes the necessary components for the assay to run. Assay run times are typically short, usually requiring less than ten (10) minutes. The assay is driven by capillary action across the nitrocellulose support, and by a “sump pad” at the distal end, to kick the reagents across the assay and capture the excess. The assay for E2 is made as follows: Commercially available Protein A-coated colloidal gold is incubated with saturating concentrations of mouse monoclonal antibodies specific for E2. The Protein A binds to the specific antibody and thereby creates a probe and reporter pair. This type of detection system is commonly used in strip tests such as home pregnancy tests. This conjugate will be dried into a conjugate application fleece. Downstream from the conjugate fleece, two spatially distinct zones will be applied and dried onto the strip using striping equipment (Kinematic Corporation Matrix 1600 Reagent Dispenser). The first line or “test” line will be a known concentration of an E2-BSA mixture. The BSA (bovine serum albumin) is present to make the estradiol adhere to the nitrocellulose. The second zone, the “control” line, will be a known concentration of goat anti-mouse antibody. The application of a negative sample (zero concentration E2) will result in a high concentration of the colloidal gold-mouse anti-E2 binding to the E2 on the test line, with the remainder binding at the control line. When E2 is present in the sample, the free E2 will bind to the anti E2 antibody-colloidal gold conjugate, preventing the conjugate from binding at the test line. This complex will pass through the test line and will be bound by the anti-mouse antibodies at the control line.

A lateral flow assay for EE2 is correspondingly prepared. The device of the invention would contain at least 4 assay chambers as follows: a first chamber for detecting E2, a second chamber for detecting EE2, and third and fourth control chambers, one for each E1 and E2.

The cartridge device 1 as described herein is designed to split or “port” the total sample volume into a sub-fraction for each assay. The relative signals from the test and control lines will then be inversely or directly proportional respectively to the concentration of E2 in the test sample.

In a preferred embodiment, between 2 and 8 distinct chemical assays are held by the MSRC.

In another preferred embodiment, between 2 and 8 identical chemical assays are held the cartridge 1. Alternatively, if cartridge 11 is being used, in a preferred embodiment, between 2 and 8 cartridges 11, each containing identical chemical assays, are joined together for simultaneous use. These embodiments assist in a statistical analysis of the accuracy of the testing results.

1. A device, for simultaneously conducting multiple assays on a liquid sample, comprising the following:
(a) a housing;
(b) an inlet in the housing for introducing the liquid sample into the housing;
(c) a diversion dam within the housing and in communication with the inlet;
(d) a diversion dam channel within the housing and in communication with the diversion dam;
(e) a first assay chamber and a second assay chamber within the housing, wherein the first assay chamber comprises a first assay for detecting the presence of an analyte in the liquid sample, and the second assay chamber comprises a second assay for detecting the presence of an analyte in the liquid sample; and
(f) a first flow channel and a second flow channel, wherein
the first flow channel is in communication with the diversion dam channel and the first assay chamber, and
the second flow channel is in communication with the diversion dam channel and the second assay chamber.

2. The device of claim 1, wherein each assay chamber further comprises an assay viewing window in the housing for reading the progress or results of the assay.

3. The device of claim 1, wherein each assay chamber further comprises an indicium viewing window in the housing for viewing a machine-readable indicium associated with the assay.

4. The device of claim 3, wherein the indicium is on the assay.

5. The device of claim 4, wherein the indicium is a bar code.

6. The device of claim 4, wherein the indicium is readable by a CCD camera or smart phone.

7. The device of claim 1, wherein the assays are lateral flow assays.

8. The device of claim 1, wherein the housing is capable of being opened and closed by a user to remove and replace the first and second assays with third and fourth assays.

9. The device of claim 1, further comprising seven or more assay chambers and seven or more flow channels.

10. A device for conducting assays on a liquid sample, comprising:
(a) a housing;
(b) an inlet in the housing for introducing the liquid sample into the housing;
(c) an assay chamber within the housing comprising an assay for detecting the presence of an analyte in the liquid sample; and
(d) means to join the device to a second device.

11. The device of claim 10, wherein the means to join the device to a second device comprises projections on two opposite and substantially parallel sides of the housing, said projections capable of interlocking the housing with housing on a second device.

12. The device of claim 11, wherein the assay chamber further comprises an assay viewing window in the housing for reading the progress or results of the assay.

13. The device of claim 11, wherein each assay chamber further comprises an indicium viewing window in the housing for viewing a machine-readable indicium on the assay.

14. The device of claim 13, wherein the indicium is a bar code.

15. The device of claim 13, wherein the indicium is readable by a CCD camera or smart phone.

16. A system for simultaneously conducting multiple assays on a liquid sample,
comprising the device according to claim 1 and a reader for reading the results of the assays in the device.

17. The system of claim 16, wherein the reader is a hand-held unit comprising a digital camera and processing hardware and software for interpreting and displaying results of the assays.

18. The system of claim 17, wherein each assay has an associated indicium that identifies the assay.

19. The system of claim 18, wherein the indicium is on the assay.

20. The system of claim 17, wherein the reader has a receptacle into which the device is inserted.

21. A method for simultaneously conducting multiple assays on a liquid sample, comprising:
(i) introducing a liquid sample into a device comprising
(a) a housing;
(b) an inlet in the housing for introducing the liquid sample into the housing;
(c) a diversion dam within the housing and in communication with the inlet;
(d) a diversion dam channel within the housing and in communication with the diversion dam;
(e) a first assay chamber and a second assay chamber within the housing, wherein the first assay chamber comprises a first assay for detecting the presence of an analyte in the liquid sample, and the second assay chamber comprises a second assay for detecting the presence of an analyte in the liquid sample; and
(f) a first flow channel and a second flow channel, wherein
the first flow channel is in communication with the diversion dam channel and the first assay chamber, and
the second flow channel is in communication with the diversion dam channel and the second assay chamber; and
(ii) reading results of each assay.

22. The method of claim 21, wherein the step of reading results is performed by a CCD camera or smart phone.

23. The method of claim 22, wherein the liquid sample is selected from the group consisting of a biological sample obtained from an animal, a biological sample from a plant, or an environmental water sample.
